(19) United States
(12) Patent Application Publication
(10) Pub. No.: US 2026/0022416 A1
(43) Pub. Date: Jan. 22, 2026

(54) Microsphere Reagent for Nucleic Acid Amplification

(71) Applicants: Shanghai SophiBiotech Co., Ltd., Shanghai (CN); Jiangsu BioPerfectus Technologies Co., Ltd, Taizhou City (CN)

(72) Inventors: Guohui ZHOU, Taizhou City (CN); Liqun FENG, Shanghai (CN); Jing YANG, Taizhou City (CN)

(21) Appl. No.: 19/263,716

(22) Filed: Jul. 9, 2025

Related U.S. Application Data
(60) Provisional application No. 63/677,237, filed on Jul. 30, 2024.

(30) Foreign Application Priority Data
Jul. 19, 2024 (CN) 202410975434.7

(51) Int. Cl. C12Q 1/686

(52) U.S. Cl. CPC C12Q 1/686

(57) ABSTRACT

The present invention provides a reagent for nucleic acid amplification, including an enzyme and a necessary reagent required for the nucleic acid amplification, where the enzyme is encapsulated by a shell formed from a thermoplastic polymer material to form a microsphere, thereby isolating the enzyme from the necessary reagent for the nucleic acid amplification. This allows nucleic acid amplification to be completed using a single-tube reagent without separate packaging of amplification components, reducing errors caused by manual operations. In the present invention, by separating the enzyme from magnesium ions, during the nucleic acid amplification, as amplification temperature increases, the polymer material encapsulating the enzyme is lysed to release the enzyme, thereby initiating the nucleic acid amplification. Additionally, the sensitivity of the nucleic acid amplification is also improved.

### CROSS-REFERENCE TO RELATED APPLICATIONS

The present invention claims priority to Chinese Prior Application No. 202410975434.7, filed on Jul. 19, 2024, and U.S. Provisional Application No. 63/677,237, filed on Jul. 30, 2024, the entire contents of which are incorporated herein by reference as part of the present invention.

### BACKGROUND OF THE INVENTION

### Field of the Invention

The present invention belongs to the technical field of molecular biology detection, and particularly relates to a preparation method and application of a reagent for nucleic acid amplification.

### Description of the Related Art

The following background introduction is only an introduction to some background knowledge and shall not constitute any limitation to the present invention.

Nucleic acid amplification or testing is a commonly used modern inspection method. By designing primers and through the action of enzymes, nucleic acids in samples can be exponentially amplified. This method can detect 1 copy of nucleic acid material in samples, featuring high test sensitivity and accuracy. Reagents required for nucleic acid amplification typically include salt ions such as magnesium ions, primers, or energy-providing substances, as well as enzymes such as polymerases. Generally, enzymes exist separately and cannot be mixed with magnesium ions, as contact between them will activate enzyme activity. When amplification is not needed, long-term contact between enzymes and magnesium will reduce enzyme activity.

A traditional solution to this problem is to store magnesium ions and enzymes in different solutions and package them separately. When amplification is needed, the two are mixed to initiate nucleic acid amplification. Another approach is lyophilization, such as preparing magnesium ions and enzymes into lyophilized powders respectively to reduce their contact. However, after lyophilization, although the contact can be reduced, the lyophilized reagent needs to be dissolved into a solution before nucleic acid amplification can be performed. In some cases requiring immediate testing, forming the lyophilized reagent into a solution within a short time period poses challenges. Moreover, extra additives need to be added during lyophilization, which may affect the efficiency of nucleic acid amplification in subsequent steps.

Aiming at the technical problems of the above traditional products, the present invention provides an improved method, offering an alternative approach to address the deficiencies of the existing traditional technologies.

### BRIEF SUMMARY OF THE INVENTION

The present invention provides a reagent for nucleic acid amplification, including a necessary reagent for nucleic acid amplification including primers, a probe, magnesium ions, and an enzyme. The enzyme is encapsulated within a microsphere made of a thermoplastic polymer material to reduce direct contact between the enzyme and the magnesium ions. Meanwhile, this reagent can be packaged in a single tube. In some implementations, the enzyme and the other necessary reagent for nucleic acid amplification are separately encapsulated in microspheres. During use, the two types of microspheres are directly mixed, and the heating inherent in nucleic acid amplification causes the microspheres to rupture, releasing contents for nucleic acid amplification. The enzyme for nucleic acid amplification is a polymerase. The reagent for nucleic acid amplification includes magnesium ions, other energy substances, or a buffer solution.

On the other hand, the present invention provides an enzyme, particularly one used in nucleic acid amplification, which is encapsulated by a thermoplastic polymer material. In this way, the enzyme is stored within a shell formed by the polymer material, so that the enzyme is relatively in an independent space, which is convenient for the storage of the enzyme and ensures the activity of the enzyme. In some implementations, the polymer material has a microporous structure, allowing the encapsulated enzyme to undergo vacuum freeze-drying. Water molecules escape from the shell through micropores, producing a lyophilized enzyme with an extremely low moisture content, thereby also facilitating enzyme storage.

In some implementations, when using PCR for nucleic acid amplification, the selected thermoplastic polymer material has a fusion or melting temperature below 100° C. At this temperature, the thermoplastic polymer material can melt into a liquid state, enabling encapsulation of the reagent for nucleic acid amplification. When a temperature drops below the melting temperature, the polymer material solidifies to form a particle.

In some implementations, the selected thermoplastic polymer material has a melting temperature below 100° C. For example, the polymer material can transition from solid to liquid at a temperature below 80° C., 60° C., or 50° C. Conversely, during the processing and encapsulation, it is more appropriate to select a material with a fusion temperature below 100° C., e.g. between 40° C. and 100° C. For example, a polymer material with a melting temperature less than 100° C. less than 90° C., less than 80° C., less than 70° C., less than 60° C., or less than 50° C. is used to encapsulate the reagent for nucleic acid amplification. Above the afore temperatures, the polymer material changes from a solid to a flowable liquid state. After encapsulation is completed, when encountering lower temperatures, the polymer material transitions from liquid to solid state, thereby forming a microsphere. When using nucleic acid amplification, such as PCR amplification, when encountering high temperature, the encapsulated reagents, such as the polymerase or the reagent for nucleic acid amplification, are released, thereby coming into contact with each other to activate the activity of the enzyme, so as to perform amplification of target nucleic acid.

In some implementations, the thermoplastic polymer material includes one or more of the following: polycaprolactone (PCL); polyvinylpyrrolidone (PVP); an ethylene-vinyl acetate copolymer (EVA); certain thermoplastic polyurethanes (TPU); polyisobutylene (PIB); certain styrene block copolymers (such as styrene-isoprene-styrene (SIS), styrene-ethylene-butylene-styrene (SEBS); polyvinyl butyral (PVB); polybutylene succinate (PBS); certain polyhydroxyalkanoates (PHA); atactic polypropylene (APP, Tg: −20° C.), petroleum resins (C5/C9); processing temperature: 70-90° C.; and a low-temperature wax-modified polymer: e.g., blends of Fischer-Tropsch wax (melting point: 60-100° C.)+polyethylene.

In some implementations, when the encapsulated reagent is the polymerase, a polyester polymer material is adopted, such as PCL, PBS, or PHB, etc. When the encapsulated reagent is a reagent for nucleic acid amplification, a polyolefin polymer material is selected, such as low-molecular-weight polyethylene (LMW-PE); an ethylene-vinyl acetate copolymer (EVA) with a high vinyl acetate (VA) content; polyisobutylene (PIB), etc.

In some implementations, the encapsulating material is a liposomal material, including one or more ionizable lipids, one or more non-ionizable lipids, one or more sterol-based lipids, and/or one or more PEG-modified lipids. Examples of suitable lipids may include, for example, phosphatidyl compounds (e.g., phosphatidylglycerol, phosphatidylcholine, phosphatidylserine, phosphatidylethanolamine, sphingolipids, cerebrosides, and gangliosides). Non-limiting examples of the ionizable lipids may include (but are not limited to) C12-200, MC3, DLinDMA, DLin-MC3-DMA, DLinkC2DMA, cKK-E12, ICE (imidazolyl), HGT5000, HGT5001, OF-02, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLin carbDAP. DLinCDAP, KLin-K-DMA, DLin-K-XTC2-DMA, SM-102, ALC-0315, HGT4003, JK-102-CA, etc., or combinations thereof. Non-limiting examples of the non-ionizable lipids may include (but are not limited to) ceramide, cephalin, cerebroside, diacylglycerol, 1,2-dipalmitoyl-sn-glycero-3-phosphoglycerol sodium salt (DPPG), 1,2-distearoyl-sn-glycero-3-phosphoethanolamine (DSPE), 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dioleoyl-sn-glycero-3-phosphocholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-sn-glycero-3-phospho-(1′-rac-glycerol) (DOPG), 1-palmitoyl-2-oleoyl-phosphatidylethanolamine (POPE), 1-palmitoyl-2-oleoyl-sn-glycero-3-phosphocholine (POPC), 1-stearoyl-2-oleoyl-phosphatidylethanolamine (SOPE), sphingomyelin, or combinations thereof. In some embodiments, the PEG-modified lipids may be poly (ethylene glycol) chains with a PEG length of 1000-5000 Da, which are covalently linked to a lipid having an alkyl chain with a length of C6-C20. Non-limiting examples of the PEG-modified lipids may include (but are not limited to) DMG-PEG₁₀₀₀, DMG-PEG₁₃₀₀, DMG-PEG₁₅₀₀, DMG-PEG₁₈₀₀, DMG-PEG₂₀₀₀, DMG-PEG₂₂₀₀, DMG-PEG₂₅₀₀, DMG-PEG₂₇₀₀, DMG-PEG 3000, DMG-PEG₃₂₀₀, DMG-PEG₃₅₀₀, DMG-PEG₃₇₀₀, DMG-PEG₄₀₀₀, DMG-PEG₄₂₀₀, DMG-PEG₄₅₀₀, DMG-PEG₄₇₀₀, DMG-PEG₅₀₀₀, ALC-0159, M-DTDAM-2000, C8-PEG, DOGPEG, ceramide PEG, and DSPE-PEG, or combinations thereof.

### Beneficial Effects

Using the system and method described above facilitates the preservation of the reagent for nucleic acid amplification and enables adoption of a single-tube reagent format, eliminating the need for separate packaging of the reagent for amplification. This simplifies operations and enhances the sensitivity of the test.

### BRIEF DESCRIPTION OF THE DRAWINGS

FIG. 1 shows curves obtained by using a QuantStudio™ 5 real-time PCR instrument for a microsphere-encapsulated enzyme provided in an embodiment of the present invention stored at room temperature for 30 days and for 0 days.

FIG. 2 shows curves obtained by using a QuantStudio™ 5 real-time PCR instrument for a microsphere-encapsulated enzyme provided in an embodiment of the present invention stored at room temperature for 30 days and a commercially available enzyme reagent.

FIG. 3 shows morphology of microspheres when a microsphere-encapsulated enzyme provided in an embodiment of the present invention is placed in an 8-tube PCR reaction strip containing an appropriate amount of pure water and stored at room temperature for 12 months.

### DETAILED DESCRIPTION OF THE INVENTION

The structures involved in the present invention or the technical terms used are further explained below. Unless otherwise specified, they shall be understood and explained according to the general terms commonly used in the art.

### Polymer Materials for Microsphere Formation

The encapsulating reagents of the present invention are primarily liquid during processing and solidify into a stable particulate state after encapsulation is complete, which facilitates the storage and protection of the reagents encapsulated within the microspheres. The materials selected need to be liquid during processing and transform into a solid state upon cooling after encapsulation is complete. When release is required, they revert to a liquid state as the temperature rises to a certain level, thereby enabling the release of the encapsulated reagents. To meet these requirements, thermoplastic polymer materials are selected. This process typically involves physical and reversible changes: melting into a flowable liquid when heated and solidifying when cooled. When the temperature rises, the polymer materials melt into a liquid state, facilitating encapsulation. As the temperature decreases, the materials transform into a solid state to form encapsulated structures. When the temperature is raised again, the polymer materials melt and release the encapsulated reagents. In the present invention, the terms “fusion” and “melting” can be used interchangeably to denote the same meaning. In some implementations, when the encapsulated reagents are reagents for nucleic acid amplification (such as the various reagents listed in Tables 8-9 of the present invention), these reagents generally do not degrade or denature at high temperatures. Thus, materials with a relatively high melting temperature can be adopted for encapsulation. However, when these materials are used for nucleic acid amplification. the maximum amplification temperature is 95-100° C. Therefore, the materials should transition from solid to liquid at around 95-100° C., though they can also melt at lower temperatures such as below 80° C., 60° C., or 50° C. Conversely, during the processing and encapsulation, it is more appropriate to select materials with a fusion temperature below 100° C. (e.g., between 40-100° C.), such as polymer materials with a melting point less than 100° C., 90° C., 80° C., 70° C., 60° C., or 50° C., to encapsulate the reagents for nucleic acid amplification. These materials are then solidified into microparticles at low temperatures (e.g., below their fusion temperature). When in use, elevating the temperature to the fusion temperature of the polymer materials releases the encapsulated reagents to participate in nucleic acid amplification. It can be understood that when the polymerase is included, the polymerase used in nucleic acid amplification (PCR) is originally extracted from strains isolated in high-temperature hot springs, which can remain non-denatured at temperatures reaching 95-100° C. When other enzymes are encapsulated, such as recombinases for isothermal amplification, the melting temperature of thermoplastic polymer materials can be selected to be lower than the protein denaturation temperature. In this way, when the temperature rises, the thermoplastic polymer materials transition from a solid to a liquid state, while the encapsulated proteases still maintain their activity.

In other words, this is directly related to the selection of the encapsulated reagents. When the encapsulated reagents do not contain reagents for nucleic acid amplification such as protein polymerases, the following polymer materials can be selected as encapsulating agents. For example, commodity thermoplastics: polyethylene (PE) such as low-density polyethylene (LDPE) with a melting point of about 105-115° C. and linear low-density polyethylene (LLDPE) with a melting point of about 120-125° C.: polyvinyl chloride (PVC) including unplasticized PVC (uPVC) with a glass transition temperature (Tg) of about 80° C. and a processing temperature of about 160-210° C. Plasticized PVC: plasticizers reduce the softening point for lower processing temperatures. Polystyrene (PS): general-purpose polystyrene (GPPS) with a glass transition temperature (Tg) of about 100° C. and a processing temperature of about 180-280° C. (no sharp melting point, softening to flow). High-impact polystyrene (HIPS): similar to GPPS but with higher impact strength. Acrylonitrile butadiene styrene (ABS): no obvious sharp melting point, a glass transition temperature (Tg) of about 105° C., and a processing temperature of about 200-250° C. (softening to flow). Polymethyl methacrylate (PMMA/acrylic): a glass transition temperature (Tg) of about 105° C. and a processing temperature of about 210-250° C. (softening to flow). Thermoplastic elastomers (TPEs): such materials exhibit rubber-like elasticity at room temperature but can melt, flow, and be processed like thermoplastics when heated. Styrene block copolymers (SBCs): styrene-butadiene-styrene (SBS) and SEBS (hydrogenated SBS). The softening/fusion temperature depends on the styrene block, typically flowing in the range of 100-200° C. Thermoplastic polyurethane (TPU): a broad fusion temperature range. The low-temperature performance is determined by the type of soft segments, while the melting point is governed by the hard segments. It typically melts in the range of 120-220° C. Bio-based/biodegradable thermoplastics: polylactic acid (PLA): a melting point of about 150-160° C. (for crystalline types with a high L-isomer content). Polyhydroxyalkanoate (PHA): such as polyhydroxybutyrate (PHB), with a melting point of about 170-180° C.; and polybutylene succinate (PBS), with a melting point of about 115° C.

The melting points of some of the above materials exceed 100° C. Blending multiple materials can lower the fusion temperature. For example, mixing with materials with low melting temperatures reduces the melting temperature, i.e., the temperature required for processing.

In some implementations, the following thermoplastic materials may also be selected. For example, polycaprolactone (PCL): a melting point of about 60° C. (extremely low). Polyvinylpyrrolidone (PVP K30): despite no fixed melting point, it has a low glass transition temperature (about 100° C.) and can be processed in aqueous solutions, making it particularly suitable for heat-sensitive proteins.

In some preferred examples, thermoplastic polymers with melting points below 100° C. may be selected. For example, polycaprolactone (PCL): a melting point of about 60° C.; low-melting-point PE: a melting point of 80-100° C. (such as ultra-low molecular weight polyethylene or specifically copolymer-modified grades), such as polyolefins; ethylene-vinyl acetate copolymer (EVA): a melting point of 70-90° C. (grades with a vinyl acetate content >28%); selected Thermoplastic Polyurethanes (TPU): a melting point of 80-100° C. (grades with high soft segment ratios, such as polyether-based TPU); polyisobutylene (PIB): a melting point of about 70° C. and a processing temperature of 90-120° C.; certain styrene block copolymers (such as SIS, SEBS): a glass transition temperature (polystyrene blocks) is 100° C., but the overall flow temperature can be as low as 80-120° C.; and polyvinyl butyral (PVB): a glass transition temperature of 60-70° C. and a processing temperature of 120-150° C.

Biodegradable thermoplastic materials may also be selected. For example, modified grades of polybutylene succinate (PBS): a melting point of 90-100° C. (the melting point reduced via copolymerization); and certain polyhydroxyalkanoates (PHA): a melting point of 80-160° C. (e.g., a melting point of PHBV copolymer can be as low as about 100° C.). Other similar thermoplastic materials may also be selected as the encapsulating materials of the present invention. For example, polymers dedicated to hot-melt adhesives: e.g., atactic polypropylene (APP, Tg: −20° C.), petroleum resins (C5/C9); processing temperature: 70-90° C.; and a low-temperature wax-modified polymer: e.g., blends of Fischer-Tropsch wax (melting point: 60-100° C.)+polyethylene.

When the encapsulated substance is protein materials such as enzymes, the following thermoplastic polymer materials may be selected. For example, polycaprolactone (PCL): a melting point of 58-60° C.; e.g., Capa 6500 by Perstorp; low-melting-point EVA: a melting point of 65-80° C.; and fatty acids: a melting point of 50-70° C. For example, a specific preparation process is as follows: PCL is heated to 70° C. until molten and then mixed and emulsified with a protein suspension. Microspheres are formed via spray chilling or melt dropping, where the mixture solidifies instantly upon contact with cold air or liquid. When the temperature increases, the polymer material melts, thereby releasing the protein to participate in nucleic acid amplification. For example, the reagents listed in Table 1 below, which may be used as enzyme-encapsulating polymer materials in this invention, are all commercially available.

TABLE 1
manufacturers and molecular weights of thermoplastic polymer materials

| | Molecular | Melting | |
|---|---|---|---|
| Grade (manufacturer) | weight (Mn) | point (° C.) | Melt index (g/10 min) |
| Capa ™ 6500 (Perstorp) | 50,000 | 58-60 | 7 (100° C., 2.16 kg) |
| Capa ™ 6800 (Perstorp) | 80,000 | 58-60 | 3 (100° C., 2.16 kg) |
| Lactel ® B6000 (DuPont) | 45,000-50,000 | 60-63 | 10-15 (80° C., 2.16 kg) |

In some implementations, thermoplastic polymer materials with a fusion temperature below 100° C. are selected.

TABLE 2
polyolefins and manufacturers

| | Representative | Melting | | Application |
|---|---|---|---|---|
| Material | grade | point (° C.) | Characteristics | scenarios |
| Low-molecular- | Epolene C10 | 80-85 | Waxy, easily | Hot-melt |
| weight polyethylene | (Westlake) | | emulsifiable | adhesives, |
| (LMW-PE) | | | | cosmetic |
| | | | | microspheres |
| EVA with a high | Evatane 42-60 | 60-65 | High flexibility, | Protein |
| VA content | (Arkema) | | good low- | encapsulation, |
| | | | temperature | shoe material |
| | | | fluidity | foaming |
| Polyisobutylene | Oppanol B10 | No melting | Tg = −70° C., | Chewing gum |
| (PIB) | (BASF) | point* | processing | base, sealants |
| | | | temperature | |
| | | | 90° C. | |

TABLE 3
polyesters and representative manufacturers

| | | Melting | |
|---|---|---|---|
| | Representative | point | |
| Material | grade | (° C.) | Characteristics |
| Polycaprolactone | Capa ™ 6500 | 58-60 | Biodegradable, |
| (PCL) | (Perstorp) | | sustained-release |
| | | | carrier |
| Polybutylene | Bionolle 1001 | 95-100 | Compostable, high in |
| succinate (PBS) | (Showa) | | strength |
| Polyhydroxybutyrate | Biomer P226 | 170-180 | Melting point ↓ after |
| (PHB) | (Biomer) | | modification (for |
| | | | copolymer PHBV, |
| | | | about 100° C.) |

TABLE 4
styrenic elastomers and representative brands

| | | Melting | |
|---|---|---|---|
| | Representative | point | |
| Material | grade | (° C.) | Characteristics |
| SIS block | Kraton D1161 | 85-95 | High viscosity, |
| copolymer | (Kraton) | | injectable at low |
| | | | temperature |
| SEBS hydrogenated | Septon 4055 | 60-65 | No solvent residue, |
| elastomer | (Kuraray) | | pharmaceutical- |
| | | | grade safety |

In some implementations, bio-based/degradable materials are also included as the encapsulating materials of the present invention.

TABLE 5
bio-based/degradable materials

| | | Melting | |
|---|---|---|---|
| | Representative | point | |
| Material | grade | (° C.) | Characteristics |
| Polylactic acid | Luminy ® L130 | 95-100 | Melting point |
| oligomer | (Total) | | reducible to 70° C. |
| (LOW-MW PLA) | | | after plasticization |
| Polycaprolactone- | Resomer ® LC 703 | 55-65 | Tunable degradation |
| lactide copolymer | (Evonik) | | rate, good |
| | | | hydrophilicity |
| Modified starch | Mater-Bi NF03 | No | Processing |
| thermoplastic | (Novamont) | melting | temperature 80-90° C. |
| | | point* | (Tg of about 60° C.) |

In some implementations, water-soluble polymer materials are also included as the encapsulating materials of the present invention.

TABLE 6
water-soluble polymer materials

| | | Fusion/melting | |
|---|---|---|---|
| Material | Representative grade | temperature | Characteristics |
| Polyvinylpyrrolidone | Kollidon ® 17 (BASF) | Softening | Rapidly soluble in cold |
| (PVP) | | point: 70-80° C. | water, protein activity |
| | | | preservation |
| Polyethylene oxide | Polyox ™ WSR N10 (Dow) | 65-70° C. | Good film-forming |
| (PEO) | | | property, sustained- |
| | | | release matrix |
| Hydroxypropyl | Metolose 60SH (Shin-Etsu) | No melting | Thermogelation |
| Methylcellulose | | point* | temperature: 50-90° C. |
| (HPMC) | | | |

TABLE 7
low-temperature hot-melt adhesive polymers

| | Processing | |
|---|---|---|
| | temperature | |
| Material | (° C.) | Characteristics |
| Atactic polypropylene | 70-85 | Strong adhesive force, rapid |
| (APP) | | curing |
| Petroleum resins | 80-95 | Tackifier, used in combination |
| (C5/C9) | | with EVA/PCL |
| Microfluidic droplet | Septon 4055 | 60° C. injection, room- |
| injection | (SEBS) | temperature curing |
| Spray chilling | Capa ™ 6500 | Melting: 70° C. → Curing: |
| | (PCL) | 5° C. |

In some implementations, liposomes may also be used as encapsulating materials. The “fusion temperature” of liposomes essentially refers to the phase transition temperature (Tm) of their phospholipid bilayer. At this temperature, the lipid membrane transforms from an ordered gel state to a disordered liquid crystal state, leading to a sudden increase in membrane fluidity and permeability, thereby releasing the encapsulated drugs or molecules. Therefore, liposomes may also serve as the encapsulating materials of the present invention. The phase transition temperature of liposomes generally ranges from 30 to 60° C. At this temperature, the encapsulated reagents can be released. In the specific preparation process, liposomes are dissolved in an organic solvent, and then nanoparticles are prepared by volatilizing the organic solvent.

Lipid nanoparticles may include one or more ionizable lipids, one or more non-ionizable lipids, one or more sterol-based lipids, and/or one or more PEG-modified lipids. Liposomes may include three or more different lipid components, and one different component of the lipids is sterol-based lipids. In some embodiments, the sterol-based lipid is imidazole cholesterol ester or “ICE” lipid (see WO2011/068810, which is incorporated herein by reference). In some embodiments, the sterol-based lipid may constitute no more than 70% (e.g., no more than 65% and 60%) of the total lipids in lipid nanoparticles (e.g., liposomes). Examples of suitable lipids may include, for example, phosphatidyl compounds (e.g., phosphatidylglycerol, phosphatidylcholine, phosphatidylserine, phosphatidylethanolamine, sphingolipids, cerebrosides, and gangliosides). Non-limiting examples of the ionizable lipids may include (but are not limited to) C12-200, MC3, DLinDMA, DLin-MC3-DMA, DLinkC2DMA, cKK-E12, ICE (imidazolyl), HGT5000, HGT5001, OF-02, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLin carbDAP, DLinCDAP, KLin-K-DMA, DLin-K-XTC2-DMA, SM-102, ALC-0315, HGT4003, JK-102-CA, etc., or combinations thereof. Non-limiting examples of the non-ionizable lipids may include (but are not limited to) ceramide, cephalin, cerebroside, diacylglycerol, 1,2-dipalmitoyl-sn-glycero-3-phosphoglycerol sodium salt (DPPG), 1,2-distearoyl-sn-glycero-3-phosphoethanolamine (DSPE), 1.2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dioleoyl-sn-glycero-3-phosphocholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-sn-glycero-3-phospho-(1′-rac-glycerol) (DOPG), 1-palmitoyl-2-oleoyl-phosphatidylethanolamine (POPE), 1-palmitoyl-2-oleoyl-sn-glycero-3-phosphocholine (POPC), 1-stearoyl-2-oleoyl-phosphatidylethanolamine (SOPE), sphingomyelin, or combinations thereof. In some embodiments, the PEG-modified lipids may be poly (ethylene glycol) chains with a PEG length of 1000-5000 Da, which are covalently linked to a lipid having an alkyl chain with a length of C6-C20. Non-limiting examples of the PEG-modified lipids may include (but are not limited to) DMG-PEG₁₀₀₀, DMG-PEG₁₃₀₀, DMG-PEG₁₅₀₀, DMG-PEG₁₈₀₀, DMG-PEG₂₀₀₀, DMG-PEG₂₂₀₀, DMG-PEG₂₅₀₀, DMG-PEG₂₇₀₀, DMG-PEG₃₀₀₀, DMG-PEG₃₂₀₀, DMG-PEG₃₅₀₀, DMG-PEG₃₇₀₀, DMG-PEG₄₀₀₀, DMG-PEG₄₂₀₀, DMG-PEG₄₅₀₀, DMG-PEG₄₇₀₀, DMG-PEG₅₀₀₀, ALC-0159, M-DTDAM-2000, C8-PEG, DOGPEG, ceramide PEG, and DSPE-PEG, or combinations thereof.

### Encapsulated Reagents

In the present invention, the reagents capable of being encapsulated by thermoplastic polymer materials to form microspheres are generally those necessary for nucleic acid amplification, which can be based on any nucleic acid amplification principle, such as isothermal amplification or temperature-variable amplification. In some implementations, the most typical example for temperature-variable amplification is reagents for PCR amplification. For instance, the necessary reagents in the nucleic acid amplification system described in Example 1 of the present invention include, for example, Tris-HCl (pH=9.0); dATP, dGTP, dCTP, dTTP; ammonium sulfate; magnesium chloride; potassium chloride; as well as primers and probes required for amplifying the target fragment. Polymerases for amplification, such as Taq enzyme, Taq DNA polymerase, Tth DNA polymerase, Pfu DNA polymerase, reverse transcription PCR (RT-PCR) specific enzymes, etc., are also included.

In some implementations, generally, the encapsulation of enzymes and other amplification reagents (excluding enzymes) are separately incorporated into two types of microspheres. For example, each microsphere contains 10 U, 5 U, or 12 U of enzyme, such that when one PCR amplification is required, adding one microsphere can meet the requirement. If liposomes are adopted to encapsulate enzymes and reagents for amplification, the number of nanoparticles can be determined through calculation. In some implementations, microdroplet methods can be adopted to form micron-scale particles. Each droplet may contain 10 U of protein, and multiple droplets can also be combined to contain a total enzyme amount of 10-20 U. 5-8 U, or 4 U. Similarly, reagents for nucleic acid amplification can also be encapsulated in an amount for one nucleic acid amplification. For example, one microsphere contains reagents for one nucleic acid amplification. It can be understood that one microsphere can include reagents for the amplification of multiple targets.

### Preparation Method

The most basic method for encapsulating reagents for nucleic acid amplification using thermoplastic materials is as follows: first, the thermoplastic material is heated to its melting temperature to transition from a solid to a liquid state, and then mixed with the reagents to be encapsulated to form a liquid droplet. When the droplet encounters a low temperature (below the melting temperature), it solidifies from a liquid to a solid state, thereby encapsulating the reagents within microspheres or particles formed by the polymer material. Specifically, the following methods are generally adopted. The most frequently adopted approach is to prepare microspheres via a microchannel method: The encapsulating material is heated to a certain temperature to form a liquid, and the reagents for nucleic acid amplification can be in a water-soluble state. Then micro-scale droplets are formed by means of microfluidics, followed by reagent encapsulation and solidification at room temperature, thus forming particulate matter.

For example, the spray chilling method can be adopted to prepare microsphere particles through the following melt emulsification steps: (1) heating the thermoplastic material (such as PCL) to 70-80° C. for melting; and (2) adding the protein-containing aqueous phase/freeze-dried powder, and homogenizing at a high speed (10,000 rpm for 2 minutes) to form a water-in-oil (W/O) emulsion, and at the same time, adding an emulsifier (Span 80) to prevent phase separation; and then atomizing the emulsion through a nozzle (the particle size is controlled by the nozzle aperture: 50-300 μm), and rapidly solidifying the atomized emulsion by cold air to form microspheres with a size of 50-300 micrometers. It can be understood that the particle size can be on a millimeter scale determined by the nozzle aperture, so that the diameter of the resulting wake flow is also on a millimeter scale. For example, microspheres with a diameter of 1-3 millimeters are prepared, and the preferred particle size is between 2.0-2.35 millimeters. Meanwhile, the content of the encapsulated protein can be controlled by adjusting equipment parameters (e.g. the activity unit “U” of the enzyme). It should be noted that the enzyme activity U is not directly related to the enzyme mass. In the nucleic acid amplification of the present invention, the amplification is primarily achieved by leveraging the activity level of the polymerase. Generally, an enzyme activity of 5 U, 10 U, or 20 U is sufficient for amplifying a single target nucleic acid. When multiple target nucleic acids are tested simultaneously, 10 U is generally adequate, and in some cases, even less is needed.

Additionally, the double emulsion-solvent evaporation method (preferred for water-soluble proteins) can be selected, which is an optimal method for water-soluble proteins. The specific method is as follows: polymer materials such as PLGA, EC, and PCL are dissolved in an organic solvent to form a solution, followed by primary emulsification (W/O): protein aqueous phase+polymer/dichloromethane solution→homogeneous formation of W/O emulsion: secondary emulsification (water-in-oil-in-water, W/O/W): pouring the W/O emulsion into an aqueous phase containing a PVA emulsifier→stirring to form a W/O/W double emulsion: and volatile solidification: continuously stirring for 6 h to volatilize the organic solvent→centrifuging to collect microspheres. The microspheres prepared in this way can have a diameter in a millimeter range or on a millimeter scale, such as 1-5 millimeters, 2-3 millimeters, etc., with a preferred particle size between 2.0-2.35 millimeters.

To adapt to large-scale production, the melt extrusion-spheronization method can also be adopted; polymer materials such as EVA, PCL, and PEG-based copolymers can be selected as the encapsulating materials. The process is as follows: melt mixing: polymer+protein powder are melt-blended in a twin-screw extruder (temperature 70-80° C.); extrusion granulating: the hot melt is extruded into strips through a porous die→the strips are cut into cylinders by rotating blades; and thermal spheronization: heating to above the material's Tg in a fluidized bed→spheronization under surface tension. The microspheres prepared in this way can have a diameter in a millimeter range or on a millimeter scale, such as 1-5 millimeters, 2-3 millimeters, etc., with a preferred particle size between 2.0-2.35 millimeters.

### Generation of Droplets

In one embodiment, the present invention provides a droplet generation device capable of producing thousands of droplets. Subsequently, a liquid containing a polymerase is discretized into thousands of individual droplets, with each droplet containing a certain amount of the polymerase, such as 5 U, 10 U, or 100 U. After droplet generation, the droplets are subjected to a temperature below the melting point of the polymer material, causing the encapsulating material to solidify and form microspheres. In one embodiment, the droplet generation device is a microfluidic platform capable of generating and discretizing a droplet of the polymerase into a large number of individual droplets. In some implementations, the droplet arrangement device of the present invention includes a droplet generation device, and the droplet generation device is in communication with the arrangement device via a microchannel.

Those skilled in the art will readily appreciate that various types and forms of droplet generation devices can be used in the present invention, provided that such devices are capable of producing droplets suitable for the intended purposes of the present invention. In one embodiment, the droplet generator is provided with inlets for introducing various droplets, such as oil or liquid polymer materials, and samples and reagents for carrying out reactions, into the droplet generator. In one embodiment, the various liquids used to generate droplets are supplied to the droplet generator through the same inlet. In one embodiment, the various liquids for generating droplets are supplied to the droplet generator through different inlets. Traditional droplet generation equipment can also be used to prepare droplets with diameters on a millimeter scale, which are then solidified into the microspheres of the present invention.

The droplet generation device of the present invention can be any structure or system capable of discretizing a liquid into a large number of droplets. In one embodiment, the droplet generation device includes, but is not limited to, a structure such as a flow-focusing structure, a cross-flow structure, a co-flow structure, a step emulsification structure, and a microchannel emulsification structure. P. Zhu and L. Wang (2017) describe some techniques for droplet generation, and their content is incorporated herein by reference in its entirety.

In one embodiment, the droplet generator is a shear-based droplet generation device that utilizes shear force to pinch fluid streams into small droplets. In one embodiment, the shear-based droplet generation device includes, but is not limited to, those composed of a cross-flow structure, a co-flow structure, and a flow-focusing structure.

In one embodiment, the droplet generation device is a surface-tension-based droplet generation device, where surface tension acts as the dominant driving force during droplet breakup. In one embodiment, the surface-tension-based droplet generation device includes, but is not limited to, those composed of a T-junction structure, a step emulsification structure, and a microchannel emulsification structure.

In one embodiment, the droplet generation device includes a droplet generation structure as described in WO2016189383A1, the content of which is incorporated herein by reference in its entirety. In one embodiment, methods capable of generating droplets can be used in the present invention to generate droplets, including but not limited to high-shear mixing, ultrasonic emulsification, high-pressure homogenization, and membrane emulsification.

In one embodiment, the droplet generation device includes a flow-focusing structure capable of compressing the flow to enhance the focusing effect. In one embodiment, the flow-focusing structure is a 2D planar flow-focusing structure.

In one embodiment, the droplet generation device includes a cross-flow structure that allows the continuous phase and the dispersed phase to intersect at an angle θ. In one embodiment, the droplet generator includes a T-shaped, Y-shaped, double-T-shaped, K-shaped, or V-shaped junction structure.

In one embodiment, the droplet generation device includes a co-flow structure in which the dispersed streamlines are sheared by the surrounding flowing continuous phase. In one embodiment, the co-flow structure is a 2D planar co-flow structure.

In one embodiment, the droplet generation device includes a step emulsification structure. In one embodiment, the droplet generation device includes a step emulsification structure combined with a parallel or perpendicular T-junction structure.

In one embodiment, the droplet generation device includes a microchannel emulsification structure.

In one embodiment, components or parts of the droplet generation structure responsible for generating droplets have hydrophobic surfaces. This can be achieved through chemical surface coating by conjugating hydrophobic groups to the surfaces of the components or parts. In one embodiment, a surfactant such as Span 80, Tween 20, Abil EM90, or a perfluoropolyether-polyethyleneoxide-perfluoropolyether triblock copolymer (PFPE-PEG-PFPE) is added to the molten polymeric thermoplastic material or aqueous phase to prevent droplet coalescence or to prevent molecules such as enzymes, DNA, or RNA from adhering to solid surfaces or water-oil interfaces.

In one embodiment, the generated droplets are emulsion droplets, not limited to a specific type of emulsion. In one embodiment, the emulsion includes but is not limited to oil-in-water, water-in-oil, and water-oil-water double emulsions.

In one embodiment, oil (also referred to as the oil phase) and a surfactant are used to generate droplets. In one embodiment, the ratio of surfactant to oil is 1-5% (by weight). In one embodiment, the oil used to generate droplets includes the above-mentioned thermoplastic polymer material in a molten liquid state. In one embodiment, the surfactant used includes, but is not limited to, Span 80, Tween 20/80, ABIL EM 90, phospholipids, and PFPE-PEG-PFPE. Baret and Jean-Christophe (2012) describe surfactants used in droplet-based microfluidics, the content of which is incorporated herein by reference in its entirety.

In one embodiment, the droplet generation device is capable of encapsulating the polymerase into water-in-oil droplets (droplet diameter of 2-3 mm) at a frequency of approximately 1 Hz to approximately 20 Hz. In one embodiment, the frequency of droplet generation is approximately 5 to 10 Hz. The size of the generated droplets is on a millimeter scale.

In some embodiments, the present invention provides a device capable of generating gel microspheres, the device including a microfluidic channel, a first inlet for introducing a polymerase solution, and a second inlet for introducing a gel solution. The first inlet and the second inlet are connected via the microfluidic channel, and at the junction thereof, the gel solution is brought into contact with a cell solution to form gel microspheres, where each of at least some of the gel microspheres includes a fixed unit of the polymerase.

The microfluidic pathway herein includes a plurality of microfluidic channels that are in fluid communication with each other. The generation of gel-encapsulated or coated polymerase droplets, or oil-phase-coated or encapsulated gel microsphere structures on a first microfluidic pathway and/or a second microfluidic pathway can be achieved by any structure in the prior art. For example, a cross-flow structure allows the continuous phase and the dispersed phase to intersect at an angle θ. In one embodiment, the droplet generator includes a T-shaped, Y-shaped, double-T-shaped, K-shaped, or V-shaped junction structure.

### Droplet Characteristics

In one embodiment, the quantity, size (e.g., diameter), volume, and emulsion type of the droplets generated or used by the present invention depend on subsequent processes or required analyses. In one embodiment, the number of generated droplets ranges from hundreds to millions.

In one embodiment, the size of the generated droplets ranges from about 5 micrometers to about 200 micrometers, or 1-5 millimeters. In one embodiment of polymerase dispersion, the size of the generated droplets is about 10 micrometers to 200 micrometers, or individual droplets are between 1-5 millimeters. In one embodiment, the generated droplets have a uniform diameter. In one embodiment, the generated droplets have a uniform diameter with a coefficient of variation of less than 5%. In another embodiment, droplets of different diameters can be generated by adjusting loading pressure. In some implementations, each droplet contains 10 U of the polymerase. For example, a solidified microsphere with a diameter of 2 millimeters contains 10 U of the polymerase.

In the subsequent preparation process of the droplets generated by the present invention, the droplets containing the polymerase are allowed to fall into a solution below the melting temperature of the polymer material, thereby solidifying to form microspheres. The solidified microspheres encapsulate reagents such as the polymerase or the necessary reagent for nucleic acid amplification.

For example, microspheres can be prepared using a microfluidic approach as follows. The microfluidic droplet injection method and microfluidic chip design can be adopted. The internal phase (aqueous phase) consists of a protein (polymerase)-containing buffer solution+0.5% sodium alginate (sodium alginate can be omitted). The external phase (oil phase) is molten PCL (80° C.). The two phases form monodisperse W/O droplets (50-200 μm or 1-5 mm in diameter) in a flow-focusing structure. For solidification by cooling, the droplets are dropped into a 4° C. PBS solution→the PCL shell solidifies to form microparticles. The aqueous phase may also include reagents for nucleic acid amplification, such as those specified in the reagent formulations listed in Tables 8-9 in the examples. The microfluidic droplet production method can be implemented using ready-made microfluidic droplet equipment. When droplets with a diameter of 1-5 mm need to be prepared, the microchannels can be set to 1×1 mm or larger. A T-junction or flow-focusing structure can be adopted. The flow rate of the oil phase is controlled and reduced to minimize shear force on the aqueous phase, allowing the oil phase sufficient time to form large droplets relying on surface tension or periodic disturbance. In the present invention, polymer materials with melting points below 100° C. are used. After melting into a liquid phase, their surface tension is extremely high, which is sufficient to form millimeter-scale droplets. Moreover, each droplet can contain enough enzyme activity units required for nucleic acid amplification, such as 10-15 U/droplet or 10-15 U/microsphere.

In one embodiment, microfluidic channels are fabricated from materials selected from silicon, glass, plastic, and polydimethylsiloxane (PDMS). In one embodiment, microfluidic channels of the same type or configuration can be used in various components of the integrated droplet microfluidic system of the present invention. In another embodiment, microfluidic channels of various types or configurations can be used in various components of the integrated droplet microfluidic system of the present invention.

In one embodiment, the droplet generator of the present invention includes two

microfluidic channels for transporting oil and one or more microfluidic channels for transporting the encapsulated reagents. In one embodiment, the actual configuration depends on the type of emulsion selected and the number of inlets required. In one embodiment, the outlet of the present invention includes one microfluidic channel with a diameter of several hundred micrometers. Droplets exiting from this outlet directly enter a low-temperature PBS solution, forming solidified microspheres.

### Detection

Detection means assaying or testing the presence or absence of a substance or a material, including but not limited to, a chemical substance, an organic compound, an inorganic compound, a metabolite, a drug, a drug metabolite, an organic tissue, a metabolite of an organic tissue, a nucleic acid, a protein, or a polymer. In addition, detection means testing the quantity of a substance or a material. Further, assay also means immunoassay, chemical assay, enzyme assay, and the like.

### Sample

Specimens tested by the detecting device of the present invention include biological fluids (for example, case fluid or clinical specimens). Liquid specimens or fluid samples may be derived from solid or semi-solid specimens, including stool, biological tissues, and food specimens. The solid or semi-solid specimens may be converted to liquid specimens by any appropriate methods, such as mixing, mashing, macerating, incubating, dissolving, or digesting the solid specimens by enzymolysis in suitable solutions, such as water, phosphate solutions, or other buffer solutions. “Biological specimens” include animal, plant, and food-derived specimens, including, for example, human or animal-derived urine, saliva, blood and components thereof, spinal fluid, vaginal secretions, sperm, stool, sweat, secretions, tissues, organs, tumors, cultures of tissues and organs, cell cultures, and media. Preferably, the biological specimen is urine; and preferably, the biological specimen is saliva. The food specimens include food-processed materials, final products, meat, cheese, wine, milk, and drinking water. The plant specimens include specimens derived from any plants, plant tissues, plant cell cultures, and media. “Environmental specimens” include specimens derived from the environment (for example, liquid specimens from lakes or other bodies of water, sewage specimens, soil specimens, groundwater, seawater, and waste liquid specimens). The environmental specimens may further include sewage or other wastewater.

The specimens herein refer to substances containing target nucleic acids, such as viruses, bacteria, fungi, and other substances from which nucleic acids can be extracted and amplified, including, for example, SARS-CoV-2, influenza virus, human papillomavirus (HPV), etc.

### PCR Nucleic Acid Amplification

Polymerase chain reaction (PCR) is a technology for amplifying target sequences under the condition of temperature variation. Amplification is performed according to the method in Table 10. When the temperature is 95° C., microspheres containing enzymes or nucleic acid reagents melt and release the reagents to participate in nucleic acid amplification. This release is slow, which improves the utilization efficiency of the enzymes and enhances the sensitivity of amplification. PCR can be divided into conventional PCR, nested PCR, multiplex PCR, hot-start PCR, real-time fluorescent quantitative PCR, digital PCR, DNA PCR, reverse transcription PCR, one-step RT-PCR, two-step RT-PCR, etc., all of which can use the reagents of the present invention for target nucleic acid amplification.

### Description of the Embodiments

The description of the embodiments of the present invention is intended to further illustrate how the present invention is implemented, and shall not constitute any limitation to the present invention. The scope of protection of the present invention is limited by the claims.

### Example 1

This example is a test comparison between the microsphere-encapsulated polymerase of the present invention and a commercially available enzyme reagent.

### 1. Information on the Commercially Available Enzyme Reagent

Selected commercially available enzyme: Robustart Taq (antibody-modified Taq polymerase), product number: E16, purchased from Zhuhai Biori Biotechnology Co., Ltd., unit: 2000 U. The Taq DNA polymerase Buffer: 10X (KL-25-06840), purchased from Shanghai Kang Lang Biological Technology Co., Ltd.

### Example 1.1: Preparation Process of Microspheres

The basic principle of the droplet generation device is as follows: the microchip of the droplet generator is made of polydimethylsiloxane (PDMS), silicon, or plastics (polycarbonate, cyclic olefin copolymer (COC)). For the fabrication of PDMS microchips, SU-8 photoresist (Microchem) was used to form a mold via photolithographic patterning on a silica substrate. The mold was used to produce PDMS replicas. The PDMS replicas were bonded to a cover glass via plasma treatment to form a PDMS chip. The surface of the chip was treated with fluorosilicate (Aquapel) to obtain hydrophobicity. A silicon chip was fabricated in a similar manner. A silicon wafer with patterned etching was bonded to a glass slide with inlets and outlets drilled by anodic bonding technology. The bonded silicon wafer was cut into individual chips. The surface of the silicon wafer was treated with fluorosilicate (Aquapel) to obtain hydrophobicity.

The device improved by Zhejiang ThunderBio Innovation Biotechnology Co., Ltd. based on the publicly disclosed patent CN217450211U was adopted in this invention to generate droplets. The microchannel was set as a 1-millimeter-wide and 1.5-millimeter-deep channel, and a T-shaped structure was used at the junction of the oil phase and water phase to prepare the microspheres of the present invention. Internal phase (aqueous phase): Taq enzyme-containing buffer+0.5% sodium alginate+0.1% Tween 80 (surface tension reduction), a flow rate of 1 microliter/min in the channel. External phase (oil phase): molten PCL (80° C.), a flow rate of 40 microliters per minute. The two phases formed monodisperse W/O droplets (particle size 2.25 mm) in the T-shaped structure. Cooling and solidification: droplets falling into 4° C. PBS→PCL shell solidification to form microparticles. Each microparticle contained about 10 U of enzyme. This method was to calculate the amount of enzyme and the number of droplets. Using 2000 U of enzyme, about 200 microspheres can be prepared, and each microsphere contains about 10 U of enzyme. Each microsphere containing 10-15U of enzyme can also be prepared. The diameter of each microsphere was about 2.25 millimeters (between 2.0 millimeters and 2.25 millimeters). To facilitate size control, a microscopic imaging device can be used at the outlet to photograph the droplet size, and the relative flow rates of the two phases can be adjusted to control the droplet size. The method described above used a modified traditional microporous approach to prepare the microsphere device of the present invention.

The above flow rates and sizes were fixed parameters selected through multiple adjustments, resulting in an average size of about 2.25 millimeters. In the implementations of the present invention, it can be understood that the size of the droplets is affected by the size of the microchannel, the properties of the polymer material itself, and the melting temperature of the organic phase which is changed to a flowable liquid phase. The aqueous phase is generally a buffer solution containing enzymes, with auxiliary reagents added to reduce surface tension. The control of the relative flow rates of the two phases using pressure, etc., can all be adjusted through a limited number of experiments according to the target particle size to be prepared to obtain the desired results. It can be understood that any method in the microsphere preparation method of the present invention can be adopted for preparation. This preparation method is a traditional existing method, and all traditional methods adopted can be realized in engineering, which is not repeated here.

### Example 1.2: Microspheres for Nucleic Acid Amplification Reagents (Excluding Polymerase)

The aqueous solution of the enzyme was replaced with a reagent solution for nucleic acid amplification (as shown in Table 8). The combination of reagents in Table 8 and primers for amplifying the target sequence (Table 9) were used to prepare droplets according to the method described in Example 1.1, and the micro-droplets were collected. Each droplet was solidified to form one reagent for nucleic acid amplification with an average particle size of 2.35 millimeters. When the amount of the reagent for nucleic acid amplification was 25 μL, the 25 μL aqueous solution was encapsulated by microspheres with a particle size of about 2.35 millimeters, and the specific number was determined until the encapsulation was completed. The encapsulating polymer material can be arbitrarily selected, such as thermoplastic polymer materials with a melting temperature lower than 100° C.

### Example 1.3: Experiment on the Effects of Different Encapsulating Materials on Polymerase and Amplification Reagent Encapsulation

Using the method described in Example 1.1, different thermoplastic polymer materials were used for the oil phase to encapsulate the polymerase and reagents for nucleic acid amplification, respectively. It was found that for the amplification effect, polyester encapsulation of the polymerase exhibited better amplification sensitivity, enzyme activity, and stability compared with polyolefin encapsulation. However, the opposite result was observed for the encapsulation of amplification reagents (excluding the polymerase). Polyolefin encapsulation of the reagents for nucleic acid amplification showed better amplification sensitivity and stability than polyester encapsulation. Thus, in a preferred implementation, polyesters are used to encapsulate the polymerase, while polyolefins are used to encapsulate the reagents for nucleic acid amplification. The specific test results are shown in Table 11.2.

2. The preparation of the nucleic acid amplification system and the amplification method are shown in Table 8 below:

TABLE 8
Preparation of a nucleic acid amplification
system (excluding the enzyme, total 25 μL)

| | |
|---|---|
| Nucleic acid amplification system | Concentration |

| Tris(hydroxymethyl)aminomethane (Tris-HCl | 120 | mM |
|---|---|---|
| PH = 9.0) | | |
| Sodium azide | 0.01% | |
| dATP | 0.4 | mM |
| dGTP | 0.4 | mM |
| dCTP | 0.4 | mM |
| dTTP | 0.4 | mM |
| Ammonium sulfate | 3 | mM |
| Magnesium chloride | 3 | mM |
| Potassium Chloride | 50 | mM |
| HPV18 Type-F | 400 | nM |
| HPV18 Type-R | 400 | nM |
| HPV18 Type-P | 300 | nM |

According to the public data from WHO and China CDC, the present invention selected primers and probes targeting the gene regions of Human Papillomavirus (HPV) Type 18. The genomic sequences of HPV were downloaded from platforms such as Virologic.org, GISAID, and GenBank of the U.S. NCBI, followed by alignment and analysis using BioEdit software to identify conserved gene regions. Specific primers and probes were then designed using Primer Express 3.0 software, as shown in Table 9 below. All primers and probes used in this example were synthesized by Shanghai Shuoying Biotechnology Co., Ltd.

TABLE 9
Sequences of primers and probes for HPV18 Type

| | |
|---|---|
| HPV18 Type-F | AAATTAGATGACACTGAAAGTT (SEQ NO: 1) |
| HPV18 Type-R | ATTGTCCCTAACGTCCTC (SEQ NO: 2) |
| HPV18 Type-P | CCATGCCGCCACGTCTAATG (SEQ NO: 2) |
| (probe) | |

### 3. Sample Information

The test samples were human papillomavirus HPV18 Type positive standards containing templates with different copy numbers.

4. The PCR amplification program is set as shown in Table 10 below:

TABLE 10
PCR amplification program

| | | | | Number of |
|---|---|---|---|---|
| Step | | Temperature | Time | cycles |
| 1 | Pre-denaturation | 95° C. | 10 min | 1 |
| 2 | Denaturation | 95° C. | 15 sec | 45 |
| | Annealing and | 56° C. | 55 sec | |
| | Extension | | | |

Fluorescence was collected during the annealing and extension in Step 2.

### 5. Test Results

The comparison test results were shown between the microsphere-encapsulated enzyme of the present invention (Example 1.1) and the commercially available enzyme, in which the nucleic acid amplification reagents were in accordance with Table 8-9 but not encapsulated. The microsphere-encapsulated enzyme provided by the present invention and the commercially available enzyme reagent (with equal enzyme contents, approximately 10 U each) were tested using a QuantStudio™ 5 real-time fluorescence PCR instrument. The CT value statistical results are shown in Tables 11.1-11.2:

TABLE 11.1
CT value statistical results

| Template | Microsphere-encapsulated | |
|---|---|---|
| concentration | enzyme (Example 1.1, PCL as | Commercially |
| (copies/mL) | an encapsulating material) | available enzyme |
| 1 × 10⁸ | 20.92 | 21.38 |
| 1 × 10⁷ | 24.35 | 25.52 |
| 1 × 10⁶ | 27.89 | 28.67 |
| 1 × 10⁵ | 31.75 | 32.57 |
| 1 × 10⁴ | 33.28 | 34.52 |
| 1 × 10³ | 35.92 | noCT (false |
| | | negative) |
| 1 × 10² | 38.29 | noCT (false |
| | | negative) |
| 1 × 10¹ | 40.25 | noCT (false |
| | | negative) |

Based on the above results, it can be known that when amplifying templates of different concentrations, the encapsulated polymerase of the present invention exhibits higher sensitivity compared with unencapsulated polymerase at the same enzyme U. The encapsulated enzyme exceeds the threshold with a relatively smaller number of cycles, leading to faster result generation. At very low template concentrations, the encapsulated enzyme can still produce detectable CT values, indicating high sensitivity. In contrast, traditional unencapsulated enzymes fail to generate CT values, suggesting no measurable result and lower sensitivity. The amplification performance of the microsphere-encapsulated enzyme provided by the present invention outperforms the commercially available enzyme reagent, particularly in terms of sensitivity. This may be because during the temperature-varying process of nucleic acid amplification, the encapsulated microspheres continuously rupture due to temperature changes, releasing the enzyme to participate in nucleic acid amplification, thereby improving the sensitivity of nucleic acid amplification, and the activity of the enzyme is utilized with the highest efficiency to improve the sensitivity of detection.

According to the method of Example 1.3, Table 11.2 compares the amplification CT values of enzyme reagents encapsulated by different microspheres (nucleic acid amplification was performed using the same method, while the enzyme was encapsulated with different encapsulating materials. The specific encapsulation process was as described in Example 1.1. The CT values corresponded to an enzyme content of 10 U and a particle size of about 2.35 millimeters).

| Template | | | | Polybutylene | | |
|---|---|---|---|---|---|---|
| concentration | | | | succinate | Oppanol | Evatane |
| (copies/mL) | PCL | PBS | PHB | (PBS) | B10 | 42-60 |
| 1 × 10³ | 35.92 | 30.35 | 30.25 | 36.25 | notCT | noCT |
| 1 × 10² | 38.29 | 32.25 | 35.25 | 38.35 | notCT | noCT |

When different materials are used to encapsulate the polymerase, the number of amplification cycles for low-concentration templates varies. Compared with PCL, when amplifying low-concentration templates, the number of cycles for PCL to reach the threshold is more than that for PBS and PHB. However, for the polymerase encapsulated in polyolefins, test results cannot be obtained at low concentrations. Although common sense suggests that polyolefins such as EVA with a high VA content are suitable for encapsulating proteins, when the encapsulated polymerase is used to perform PCR nucleic acid amplification, the effect of polyolefins as encapsulating materials does not seem to be superior to that of polyesters. Similarly, the research team of the present invention also attempted to use some materials listed in Table 4-7 to encapsulate the polymerase and test the performance of the encapsulated polymerase. The overall results show that the PCR amplification effect of the encapsulated polymerase is better than that of the unencapsulated one. However, through horizontal comparison, polyesters adopted exhibit better effects, especially higher sensitivity. Similarly, attempts were also made to encapsulate the reagents for nucleic acid amplification using the materials listed in Table 4-7. Although the PCR amplification effect is better than that of the unencapsulated ones, olefins show the best performance, enabling high sensitivity (specific experimental data are omitted).

### Example 2

This example is the activity evaluation of microsphere-encapsulated enzymes with different particle sizes in the present invention. 1. Using the preparation method in Example 1.1 (with PBS as the encapsulating material), 3 batches of microspheres encapsulating the enzyme with different particle sizes were prepared, with particle sizes of 1.7-1.85 millimeters, 1.85-2.0 millimeters, and 2.0-2.35 millimeters, respectively. 2. Enzyme activity tests were conducted on 3 batches of microspheres encapsulating the enzyme with 3 different particle sizes (the enzyme mass was equal, but the particle sizes were different), as shown in Table 12 below:

TABLE 12
Enzyme activity test

| | Enzyme activity | Enzyme activity | Enzyme activity |
|---|---|---|---|
| | (U/particle) for | (U/particle) for | (U/particle) for |
| | 1.70-1.85 millimeter | 1.85-2.0 millimeter | 2.0-2.35 millimeter |
| Batch | particle size | particle size | particle size |
| Lot1 | 10.86 | 14.77 | 18.46 |
| Lot2 | 10.85 | 14.76 | 18.45 |
| Lot3 | 10.92 | 14.86 | 18.57 |

The enzyme activity test results of the 3 batches of microsphere-encapsulated enzymes with the above 3 different particle sizes show that the microsphere-encapsulated enzyme with a particle size of 2.0-2.35 mm has the highest activity. This indicates that the activity of the enzyme encapsulated in microspheres with different particle sizes is significantly correlated with the particle size, which may be caused by the influence of the microenvironment in the particles on the enzyme activity. This directly affects the efficiency and sensitivity of PCR nucleic acid amplification.

### Example 3

1. Three batches of microsphere-encapsulated enzymes with particle sizes ranging from 2.0 to 2.35 mm were prepared (Example 1.1, the encapsulating material was PHB).

2. Tests were conducted using a QuantStudio™ 5 real-time PCR instrument with the amplification system as shown in Example 1. Each batch was added with a template at a copy number of 1×10⁶ copies/ml, and 10 tests were performed for each batch. Ten tests were conducted, and the statistical data of the precision tests are shown in Table 13.

TABLE 13
Statistical data of precision tests

| | Batch | | |
|---|---|---|---|
| Number of tests | Lot1 | Lot2 | Lot3 |
| 1 | 27.03 | 27.01 | 27.12 |
| 2 | 27.02 | 26.99 | 26.98 |
| 3 | 26.96 | 27.03 | 27.15 |
| 4 | 26.85 | 26.80 | 26.88 |
| 5 | 26.89 | 27.01 | 27.16 |
| 6 | 26.93 | 27.03 | 27.13 |
| 7 | 27.15 | 26.72 | 27.03 |
| 8 | 26.98 | 27.12 | 27.10 |
| 9 | 27.03 | 26.85 | 27.13 |
| 10 | 27.08 | 27.08 | 27.19 |
| Average CT | 26.99 | 26.96 | 27.09 |
| value | | | |
| Standard | 0.09 | 0.13 | 0.10 |
| deviation | | | |
| CV | 0.31% | 0.46% | 0.46% |

The above precision evaluation results indicate that the coefficient of variation (CV) values of the microsphere-encapsulated enzymes in the 3 batches provided by the present invention are all less than 5%, and the precision performance test is qualified.

### Example 4

This example is a test comparison between the microspheres (with a particle size of 2.0-2.35 mm) prepared in the present invention and a commercially available kit.

### 1. Information on Samples and the Commercially Available Kit

The sample was a national reference product of commercially available monkeypox virus nucleic acid detection reagents (purchased from GeneWell Biotechnology (Shenzhen) Co., Ltd.). DNA was extracted using a nucleic acid extraction kit (magnetic bead method), and the extraction kit was purchased from Jiangsu Bioperfectus Technologies Co., Ltd., with the product number: SDK60104.

The selected commercially available kit was a monkeypox virus nucleic acid assay kit (fluorescent PCR method), with the product number: YJC70115NW, purchased from Jiangsu Bioperfectus Technologies Co., Ltd. (the enzyme dosage was 10 U).

### 2. Microsphere Preparation

The amplification solution and polymerase enzyme in the monkeypox virus nucleic acid assay kit (fluorescent PCR method) with the product number YJC70115NW were respectively prepared into microspheres (particle size: 2.0-2.35 mm). The specific preparation method referred to the microfluidic control preparation method in Example 1.1, in which the polymerase was encapsulated with PCL (10 U of polymerase), and the nucleic acid amplification solution was encapsulated with polyisobutylene (PIB, Oppanol B10) (particle size: 2.0-2.35 mm).

### 3. Test Comparison

The microspheres provided by the present invention and the commercially available kit (YJC70115NW) were used to test the positive reference products in the national reference products of monkeypox virus nucleic acid detection reagents respectively by using a QuantStudio™ 5 real-time PCR instrument.

### 4. Test Results

The microspheres of the present invention and the commercially available reagent were tested by using a QuantStudio™ 5 real-time PCR instrument, and the PCR amplification results are shown in Table 14. The data statistical results are shown in the following table:

TABLE 14
Statistical data of comparison tests

| Template | | | | |
|---|---|---|---|---|
| concentration | Microsphere | | Commercially available reagent | |
| (copies/mL) | FAM | VIC | FAM | VIC |
| 10⁶ | 23.82 | 24.21 | 23.92 | 24.15 |
| | 23.91 | 24.13 | 23.81 | 24.19 |
| | 24.10 | 24.32 | 24.15 | 24.22 |
| 10⁵ | 27.22 | 27.89 | 27.12 | 27.99 |
| | 27.10 | 27.90 | 27.24 | 27.91 |
| | 27.35 | 28.11 | 27.32 | 28.01 |
| 10⁴ | 30.28 | 30.54 | 30.54 | 30.76 |
| | 30.19 | 30.49 | 30.49 | 30.78 |
| | 30.24 | 30.45 | 30.45 | 30.82 |
| 10³ | 34.51 | 35.22 | NoCt | NoCt |
| | 34.42 | 35.23 | NoCt | NoCt |
| | 34.63 | 35.41 | NoCt | NoCt |

Through the above comparison test results, it is shown that the test performance of the microspheres prepared in the present invention (with amplification solution and enzyme encapsulated into microspheres) is superior to that of the commercially available reagent, with better sensitivity. When at low concentrations, the present invention can still yield test results, while the unencapsulated nucleic acid amplification solution and polymerase fail to produce test results.

### Example 5

This example is a test comparison between the full-reagent microspheres prepared in the present invention and a commercially available kit.

### 1. Information on the Commercially Available Kit

The selected commercially available kit is the Mycoplasma pneumoniae nucleic acid assay kit (fluorescent PCR method), with the product number: YJB20109N, purchased from Jiangsu Bioperfectus Technologies Co., Ltd. (the enzyme concentration: 10 U).

### 2. Preparation of Full-Reagent Microspheres

The PCR amplification components (amplification solution, enzyme, and detection solution) of the Mycoplasma pneumoniae nucleic acid assay kit (fluorescent PCR method) with the product number YJB20109N were prepared into microspheres. The specific preparation method referred to the microfluidic control preparation method in Example 1.1, in which the polymerase was encapsulated with PCL (10 U of polymerase, particle size: 2.0-2.35 mm), and the nucleic acid amplification and detection solution was encapsulated with polyisobutylene (PIB, Oppanol B10) (particle size: 2.0-2.35 mm).

### 3. Test Comparison

The full-reagent microspheres provided by the present invention and the commercially available kit (YJC70115NW) were tested using a QuantStudio™ 5 real-time PCR instrument.

### 4. Test Results

The full-reagent microspheres of the present invention and the commercially available reagent were tested using a QuantStudio™ 5 real-time PCR instrument, and the statistical data are shown in the following table:

TABLE 15
Statistical data of comparison tests

| Template | | | Commercially available | |
|---|---|---|---|---|
| concentration | Full-reagent microsphere | | reagent | |
| (copies/mL) | FAM | VIC | FAM | VIC |
| 2500 | 25.41 | 26.56 | 25.51 | 26.61 |
| | 25.35 | 26.61 | 25.49 | 26.69 |
| | 25.37 | 26.64 | 25.56 | 26.65 |
| 250 | 29.54 | 29.76 | 30.16 | 30.37 |
| | 29.46 | 29.69 | 30.19 | 30.42 |
| | 29.51 | 29.65 | 30.23 | 30.36 |
| 25 | 32.43 | 32.66 | 32.62 | 32.79 |
| | 32.38 | 32.71 | 32.59 | 32.76 |
| | 32.41 | 32.64 | 32.56 | 32.81 |
| 10 | 36.23 | 36.43 | NoCt | 36.86 |
| | 36.27 | 36.52 | NoCt | NoCt |
| | 36.26 | 36.51 | NoCt | NoCt |
| NC (control) | NoCt | NoCt | NoCt | NoCt |
| | NoCt | NoCt | NoCt | NoCt |
| | NoCt | NoCt | NoCt | NoCt |

Through the above comparison test results, it is shown that the test performance of the full-reagent microspheres of the present invention is superior to that of the commercially available reagent, with better sensitivity.

### Example 6

This example is the stability evaluation of the microsphere-encapsulated enzyme of the present invention.

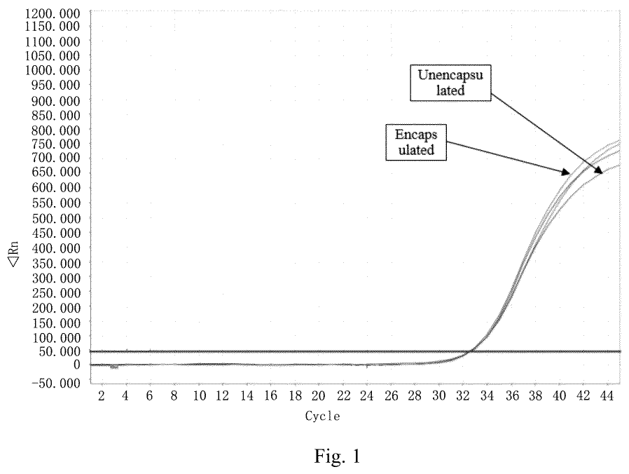

1. The microsphere-encapsulated enzyme of the present invention (with a particle size of 2.0-2.35 mm) was stored at room temperature for 30 days and 0 days, and tested using a QuantStudio™ 5 real-time PCR instrument with the amplification system as shown in Example 1. The PCR amplification results are shown in FIG. 1. PCL was used as the encapsulating material for the polymerase.

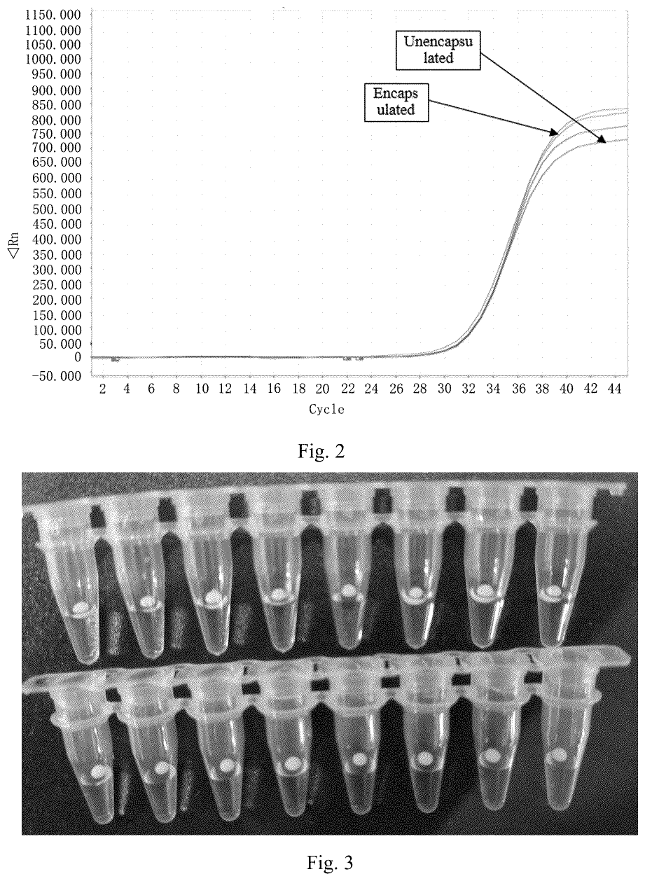

2. The microsphere-encapsulated enzyme of the present invention stored at room temperature for 12 months and the commercially available enzyme reagent (Robustart Taq (antibody-modified Taq enzyme), product number: E16, purchased from Zhuhai Biori Biotechnology Co., Ltd.) were tested using a QuantStudio™ 5 real-time PCR instrument with the amplification system as shown in Example 1. The PCR amplification results are shown in FIG. 2. The above stability evaluation results show that the microsphere-encapsulated enzyme provided by the present invention has stable performance after being stored at room temperature for 12 months.

### Example 7

This example is the water-soluble stability evaluation of the microsphere-encapsulated enzyme of the present invention.

1. The microsphere-encapsulated enzyme of the present invention (Example 1.1, with PCL as the encapsulating material and a particle size of 2.0-2.35 mm) was placed into an 8-tube PCR reaction strip, respectively. An appropriate amount of pure water was added to the reaction strip, and the 8-tube PCR reaction strip was stored at room temperature for 12 months. Visual observation showed no change in the morphology of the microspheres, as shown in FIG. 3.

2. After the 8-tube PCR reaction strip containing the microsphere-encapsulated enzyme was stored at room temperature for 12 months, the microspheres were gently removed with forceps and the surface moisture was drained.

3. The microsphere-encapsulated enzyme and the commercially available enzyme reagent (Robustart Taq (antibody-modified Taq enzyme), product number: E16, purchased from Zhuhai Biori Biotechnology Co., Ltd.) were tested using a QuantStudio™ 5 real-time PCR instrument with the amplification system as shown in Example 1. The CT value statistical results are shown in Table 16 below:

TABLE 16
CT value statistical results

| Template | Microsphere- | Commercially |
|---|---|---|
| concentration | encapsulated | available |
| (copies/mL) | enzyme | enzyme |
| 1 × 10⁸ | 20.38 | 21.32 |
| 1 × 10⁷ | 24.23 | 25.26 |
| 1 × 10⁶ | 27.32 | 28.36 |
| 1 × 10⁵ | 31.71 | 45.65 |

The above water-soluble stability evaluation results show that the microsphere-encapsulated enzyme provided by the present invention, when placed in an 8-tube PCR reaction strip containing an appropriate amount of pure water and stored at room temperature for 12 months, exhibits stable water solubility and good amplification performance. Compared with conventional unencapsulated enzymes, the activity of the encapsulated enzyme remains good, while the activity of the unencapsulated enzyme may decrease, resulting in more CT cycles, indicating that the decrease in enzyme activity is the cause.

### Example 8

This example provides a method for preparing a lyophilized reagent of a microsphere-encapsulated enzyme.

Some polymer materials have a microporous structure, allowing the encapsulated enzyme to undergo vacuum freeze-drying. Water molecules escape from the shell through micropores, producing a lyophilized enzyme with an extremely low moisture content, thereby also facilitating the storage of the microsphere-encapsulated enzyme and extending its shelf life.

Specifically, the method included the following steps:
1. preparing a microsphere-encapsulated enzyme according to the method in Example 1, using PCL as an encapsulating material (particle size: 2.0-2.35 millimeters).
2. packaging microsphere-encapsulated enzyme particles into an 8-tube PCR reaction strip.
3. placing the 8-tube PCR reaction strip into a vacuum freeze-dryer for freeze-drying.
4. when the vacuum freeze-dryer was operating normally, setting freeze-drying program parameters as shown in Table 17.

TABLE 17
Lyophilization program parameter settings

| | | | |
|---|---|---|---|
| Step | Temperature | Vacuum degree | Time |

| | | | | | |
|---|---|---|---|---|---|
| Pre-freezing | 0° | C. | | 1 | h |
| | −47° | C. | | 30 | min |
| | −47° | C. | | 2 | h |
| Sublimation | −40° | C. | 150 μbar | 30 | min |
| drying | −35° | C. | 150 μbar | 2 | h |
| | −32° | C. | 150 μbar | 5 | h |
| Analytical | 25° | C. | Ultimate | 1.5 | h |
| drying | | | vacuum | | |
| | 25° | C. | Ultimate | 5 | h |
| | | | vacuum | | |

5. after freeze-drying, strictly controlling the temperature and humidity of the environment where the freeze-dryer was located, and once the temperature and humidity of the environment met the requirements, opening a freeze-dryer chamber door and removing the lyophilized reagent in the 8-tube PCR reaction strip.
6. sealing and packaging the lyophilized reagent of the microsphere-encapsulated enzyme particles, and performing an accelerated stability test at 37° C. for 60 days before testing its performance.
7. conducting performance testing of the lyophilized reagent of the microsphere-encapsulated enzyme particles.

The lyophilized reagent of the microsphere-encapsulated enzyme and the commercially available enzyme reagent (Robustart Taq (antibody-modified Taq enzyme), product number: E16, purchased from Zhuhai Biori Biotechnology Co., Ltd.) were tested using a QuantStudio™ 5 real-time PCR instrument with the amplification system as shown in Example 1. The CT value statistical results are shown in Table 17 below:

TABLE 17
CT value statistical results

| Template | Microsphere- | Commercially |
|---|---|---|
| concentration | encapsulated | available |
| (copies/mL) | enzyme | enzyme |
| 1 × 10⁸ | 20.18 | 21.32 |
| 1 × 10⁷ | 25.13 | 25.26 |
| 1 × 10⁶ | 27.22 | 28.36 |
| 1 × 10⁵ | 31.21 | 32.65 |

The above results indicate that the lyophilized reagent of the microsphere-encapsulated enzyme prepared in this example exhibits good test performance, and the test results can be obtained with fewer amplification cycles.

All patents and publications mentioned in the specification of the present invention indicate that these are disclosed techniques in the art and can be used by the present invention. All patents and publications cited herein are likewise listed in the references as if each publication is specifically and separately referenced. The present invention described herein may be implemented in the absence of any one or more elements, and one or more limitations, which are not specifically stated herein. For example, in each instance herein, the terms “comprising/including,” “consisting essentially of,” and “consisting of” may be replaced by either of the other 2 terms. The term “a/an” here merely means “one”, but does not exclude including 2 or more instead of including only one. The terms and expressions employed herein are descriptive and are not limited thereto, and there is no intention herein to indicate that the terms and interpretations described herein exclude any equivalent features, but it can be noted that any appropriate changes or modifications can be made within the scope of the present invention and claims. It can be understood that the embodiments described in the present invention are preferred embodiments and features, and any person skilled in the art can make some modifications and changes based on the essence of the description of the present invention. These modifications and changes are also considered to be within the scope of the present invention and the scope limited by the independent claims and the dependent claims.

1. A reagent for target nucleic acid amplification, comprising: an enzyme and a necessary reagent required for the nucleic acid amplification, wherein the enzyme is encapsulated by a shell formed from a thermoplastic polymer material to form a microsphere, thereby isolating the enzyme from the necessary reagent for the nucleic acid amplification.

2. The reagent according to claim 1, wherein the thermoplastic polymer material is capable of changing from a solid state to a liquid state at a melting temperature thereof or higher, and is capable of changing from a liquid state to a solid state at a temperature lower than the melting temperature thereof.

3. The reagent according to claim 2, wherein the polymer material encapsulating the enzyme has the melting temperature below 100° C.

4. The reagent according to claim 3, wherein the melting temperature is below 95-60° C.

5. The reagent according to claim 4, wherein the enzyme is an enzyme for the nucleic acid amplification, such as a nucleic acid polymerase, a cleavage enzyme, or an enzyme used in gene editing.

6. The reagent according to claim 5, wherein the necessary reagent for the nucleic acid amplification is also encapsulated by a shell formed from a thermoplastic polymer material to form a microsphere containing the necessary reagent for the amplification.

7. The reagent according to claim 6, wherein the polymer material encapsulating the necessary reagent for the nucleic acid amplification is a polyolefin polymer material.

8. The reagent according to claim 7, wherein the olefin polymer material comprises one or more of low-molecular-weight polyethylene (LMW-PE), an ethylene-vinyl acetate copolymer (EVA) with a high vinyl acetate (VA) content, and polyisobutylene (PIB).

9. The reagent according to claim 8, wherein the polymer material encapsulating the polymerase is a polyester polymer material.

10. The reagent according to claim 9, wherein the polyester polymer material comprises one or more of polycaprolactone (PCL), polybutylene succinate (PBS), or polyhydroxybutyrate (PHB).

11. The reagent according to claim 5, wherein the microsphere has a particle size of 2.0-2.35 millimeters.

12. The reagent according to claim 1, wherein the thermoplastic polymer material is a polymer selected from one or more of the following polymers: polyolefins, polyesters, polyurethanes, acrylic acids, and polysulfones.

13. The reagent according to claim 12, wherein the polyolefins comprise polyethylene (PE) or polypropylene (PP).

14. The reagent according to claim 13, wherein the polyesters comprise polyethylene terephthalate (PET).

15. The reagent according to claim 1, wherein the thermoplastic polymer material comprises a styrenic elastomer, comprising a styrene-isoprene-styrene (SIS) block copolymer or a hydrogenated styrene-ethylene-butylene-styrene (SEBS) elastomer.

16. The reagent according to claim 1, wherein the thermoplastic polymer material comprises a biodegradable polymer material, comprising low-molecular-weight polylactic acid (LOW-MW PLA), a polycaprolactone-lactide copolymer, and a modified starch thermoplastic.

17. The reagent according to claim 1, wherein the thermoplastic polymer material comprises a water-soluble polymer material, comprising polyvinylpyrrolidone (PVP), polyethylene oxide (PEO), and hydroxypropyl methylcellulose (HPMC).

18. The reagent according to claim 1, wherein the thermoplastic polymer material is a liposomal material.

19. The reagent according to claim 1, wherein the necessary reagent for the nucleic acid amplification comprises Tris-HCl, KCl, MgCl₂, a deoxynucleoside triphosphate (DNTP), and a primer sequence for amplifying a target nucleic acid.

20. The reagent according to claim 1, wherein the nucleic acid amplification is PCR amplification.
